(19) United States
(12) Patent Application Publication
(10) Pub. No.: US 2014/0331383 A1
(43) Pub. Date: Nov. 13, 2014

(54) EYE MASK FOR SLEEPING

(71) Applicant: Ann Bially, Brooklyn, NY (US)

(72) Inventor: Ann Bially, Brooklyn, NY (US)

(21) Appl. No.: 14/254,974

(22) Filed: Apr. 17, 2014

Related U.S. Application Data
(60) Provisional application No. 61/821,812, filed on May 10, 2013.

(51) Int. Cl. A61F 9/04

(52) U.S. Cl. CPC A61F 9/04
USPC 2/173

(57) ABSTRACT

An eye mask is provided that is comprised of a fabric structure with first and second concave shapes that fit over the periorbital region of each eye and which protects the eyes while sleeping.

### CROSS-REFERENCE TO RELATED APPLICATION

This application claims the benefit of Provisional application No. 61/821,812 filed on May 10, 2013, which application is hereby incorporated by reference herein in its entirety.

### BACKGROUND

Various types of eyewear have been used in the past to protect the eye. However, while providing at least some protection under some circumstances, many of these have been too uncomfortable to wear while sleeping. Eyewear also has been made specifically for sleeping. For example, many airlines distribute eye masks to passengers to help them shut out light from their eyes and thus aid the passengers in sleeping during flights. These eye masks generally do not fit well. They also can press against the eyelid which then transfers the pressure to the eyeball where it can cause harmful increases in the pressure in the eye (i.e., increased intraocular pressure) that can cause damage to the eye over time and exacerbate eye disorders like glaucoma. These eye masks can also be uncomfortable and disorder make-up and hair.

Eye masks used for sleeping having eyecups that do not rest against the eye have also been tried. However, these particular eye masks have soft cups that can be pressed into the eyes and thus they afford little or no protection to the eye. Other eyewear has been used to protect the eyes of comatose patients and to keep their eyes closed by pressing against the eyelid. Like the airline eye masks, this eyewear can also cause harmful increases in the pressure in the eye.

To protect the eye and avoid harmful increases in the pressure in the eye, eye protection devices have been developed in the past that attempt to avoid contact with the eyelid and eyelashes and to also avoid harm to the eye from contact with other objects (e.g., mattresses, sheets, blankets, pillows, fingers, hands, arms). For example, a shield with rubber cushioning around the perimeter that is taped to the face to cover an eye has been tried but been found to be unacceptable for many people because it was too uncomfortable and/or skin reactions to the tape adhesive occurred after a few days.

U.S. Pat. No. 6,155,261 describes eye protection apparatus. No eye mask is described in that patent. Instead, goggles, apparatus to force people to sleep on their backs and to avoid head movement, conventional eye glasses, and helmets are described that appear to be neither comfortable nor lend themselves to long term use while sleeping.

Accordingly, there is a need for improved protective eyewear for sleeping that potentially can be used over the long term.

### SUMMARY

To avoid the problems of the eyewear of the past, an eye mask for sleeping has been invented that uses a fabric structure. The eye mask is preferably shaped and fitted with internal concave support structures that fit over the left and right eyes, covering the nose bridge, the upper cheekbones to the lower brow, and wrapping onto the temples, herein referred to as the periorbital regions. A strap structure extending from the edges of the eye mask at the temples to around the back of the head holds the eye mask in place.

The eye mask can be made to avoid contacting the eye, eyelid, and eyelashes with the eye mask or other objects while sleeping. The eye mask can also be made to protect the eye from objects pressing against it, thereby protecting the eyes from acute increases in intraocular pressure. The eye mask can be made to be comfortable, lightweight, light blocking and to avoid irritating the wearer. Thus, the eye mask can be potentially comfortable enough to be used every night by a wearer for a long term (e.g., a week or more).

The invention and its different embodiments and features are further described in the drawings, descriptions and claims below.

### BRIEF DESCRIPTION OF THE DRAWINGS

The invention herein will be further characterized with reference to the drawings, wherein:

FIG. 1 is a perspective view of an embodiment of an eye mask;

FIG. 2 is a perspective view of an embodiment of an eye mask;

FIG. 3 is a perspective view of embodiments of concave support structures;

FIG. 4 is a perspective view of an embodiment of a left concave support structure;

FIG. 5 is a perspective view of embodiments of left and right concave support structures;

FIG. 6 is a perspective view of embodiments of left and right concave support structures;

FIG. 7 is a perspective view of embodiments of left and right concave support structures;

FIG. 8 is a perspective view of embodiments of concave support structures;

FIG. 9 is a perspective view of an embodiment of an eye mask being worn on a head;

FIG. 10 is a sectional view of the embodiment of an eye mask being worn on a head of FIG. 9.

### DETAILED DESCRIPTION

In the following detailed description, reference is made to the drawings and to additional specific exemplary embodiments of the invention that may be practiced. It is to be understood that other embodiments and other combinations of embodiments can be used and structural and functional changes can be made without departing from the scope of the present invention.

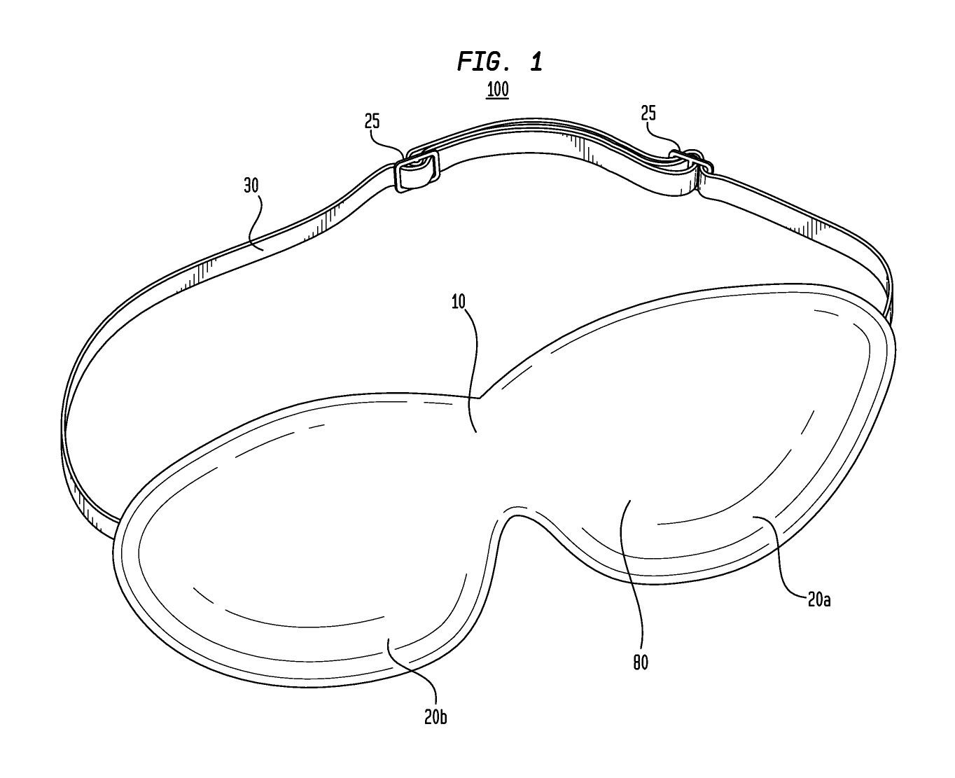

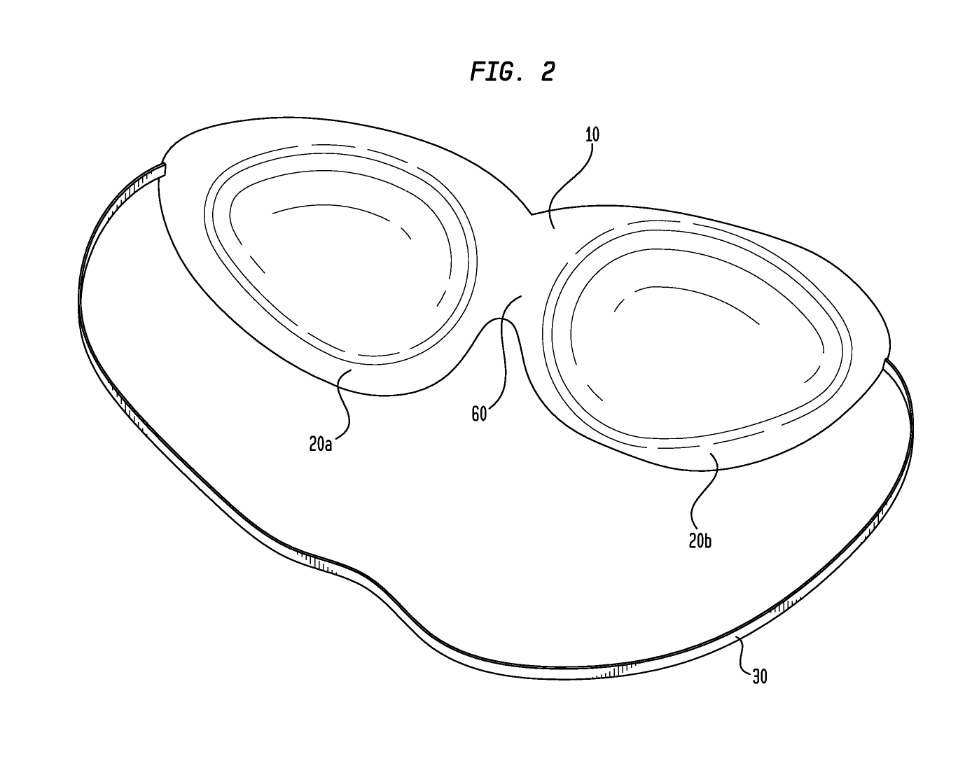

An eye mask for sleeping can be made that has the shape shown, for example, in FIG. 1. An eye mask 100 can be comprised of a fabric structure 10 that is shaped to fit over the periorbital regions of a wearer. The fabric structure can also include a strap structure 30 that holds the eye mask on the wearer's head. The fabric structure can have a first concave shape 20a (i.e., “concave” with respect to the inside of the eye mask that faces the wearer) that fits over the left periorbital region of the wearer's face and a second concave shape 20b (i.e., “concave” with respect to the inside of the eye mask that faces the wearer) that fits over the right periorbital region of the wearer's face. A perspective front view (i.e., looking at the outside of an eye mask 80 that would be away from a wearer's face if the eye mask was on the wearer's head) of an embodiment of an eye mask is shown in FIG. 1 and a perspective back view (i.e., looking at the inside of the mask 60 that would be towards the wearer's face if the eye mask was on the wearer's head) of an embodiment of an eye mask is shown in FIG. 2.

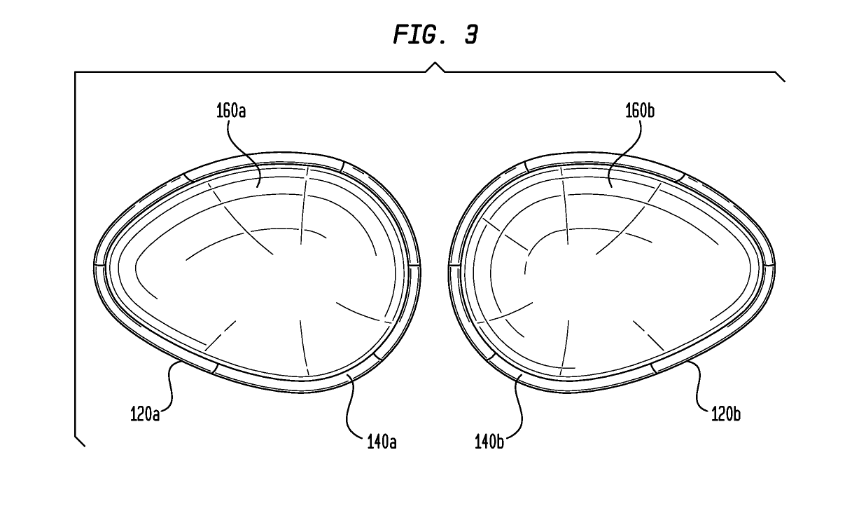

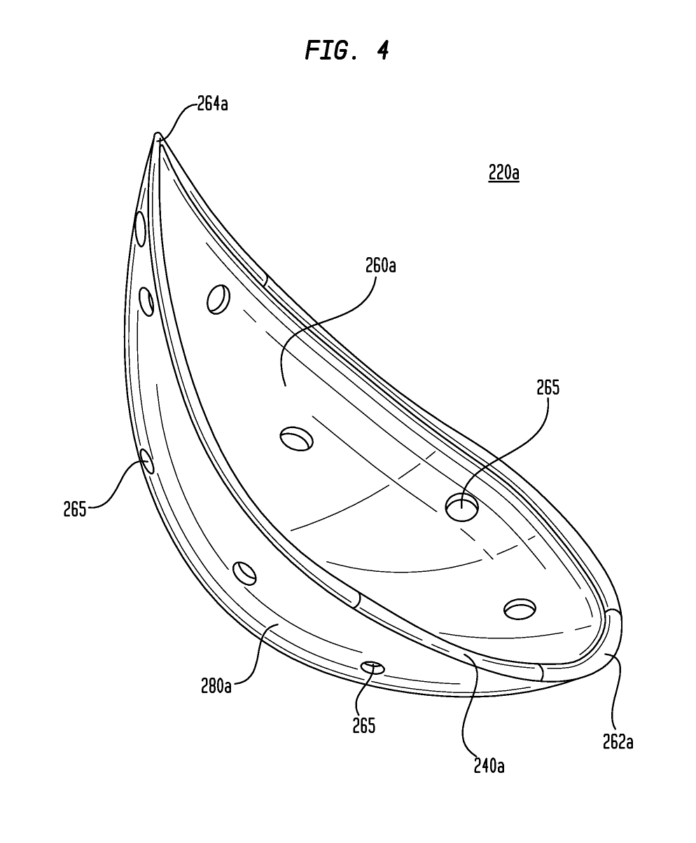

The fabric structure covers the wearer's nose bridge and is contoured to fit over the periorbital region of each eye. The eye mask preferably contains a first concave support structure that is shaped to fit inside the first concave shape of the fabric structure and over the left periorbital region of a wearer and rest on the wearer's nose, brow, cheek, and temple on the left side of the face. A second concave support structure is also preferably added and shaped to fit inside the second concave shape of the fabric structure and over the right periorbital region of the wearer and rest on the wearer's nose, brow, cheek and temple on the right side of the face. An example of a first and second concave support structure for the left eye 120a and right eye 120b, respectively, is shown in FIG. 3. The inside view 160a, 160b of the first and second concave support structures, and an optional rounded edge 140a, 140b are also shown in FIG. 3. A side view of an embodiment of a first concave support structure for the left eye 220a that contains optional ventilation holes 265 is shown in FIG. 4.

The first concave structure 220a as shown in the embodiment has an inside surface 260a and an outside surface 280a, with a portion nearest the nose 262a and a portion nearest the ear 264a, when the first concave structure is in an eye mask on a wearer's head. An optional rounded edge 240a is also shown in FIG. 4.

The fabric structure should not contact the eyelid or eyelashes when the eye mask is worn. In one embodiment, this avoidance of contact is accomplished by the fabric structure having a first concave shape that fits over the left periorbital region of a wearer and a second concave shape that fits over the right periorbital region, creating a distance between the fabric structure and the eyelid and eyelashes. These first and second concave shapes can be molded into the fabric structure or formed by other means. In another embodiment, this avoidance of contact is accomplished by attaching or joining the fabric structure to one or more surfaces of the first and second concave structures, thereby creating a distance between the fabric structure and the eyelid and eyelashes.

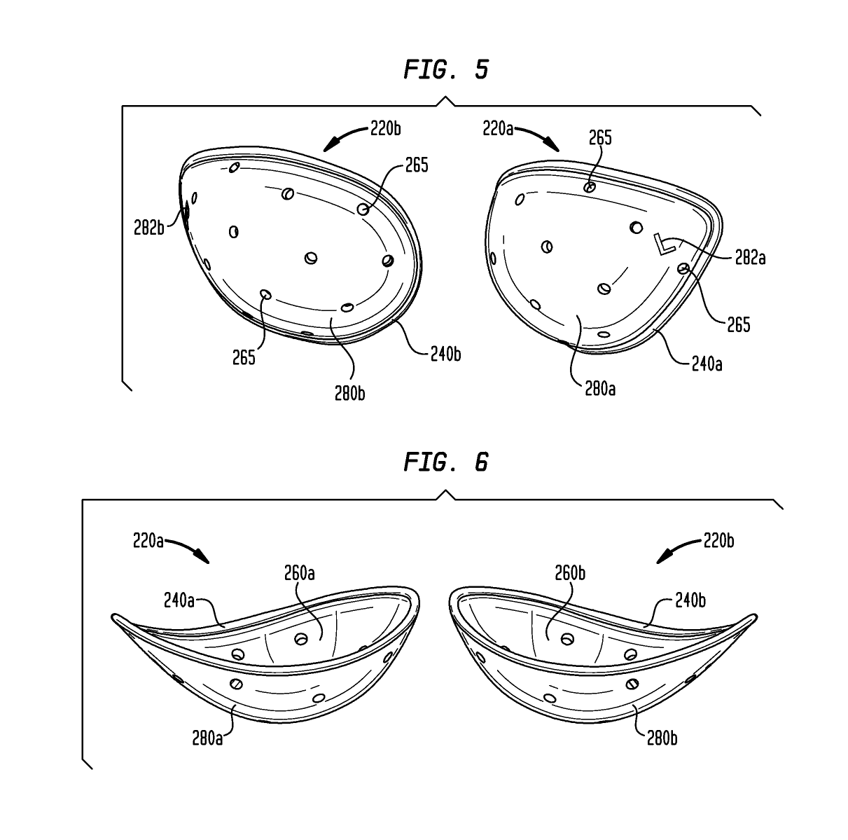

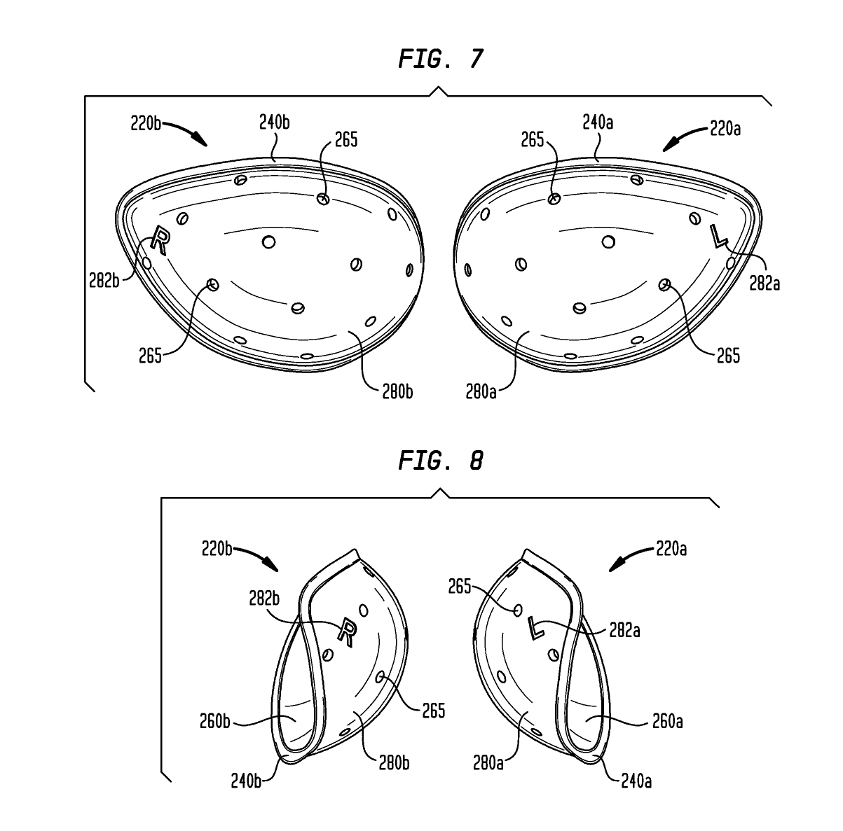

Different views of embodiments of a first concave structure 220a and a second concave structure 220b are shown in FIG. 5, FIG. 6, FIG. 7 and FIG. 8, including the inside surface 260a, 260b and the outside surface 280a, 280b, and an optional rounded edge 240a, 340b. Optional ventilation holes 265 are also shown.

When worn, the eye mask can be made to not contact or put pressure on the wearer's eyes. The eye mask can also be made to avoid objects (e.g., mattresses, sheets, blankets, pillows, fingers, hands, arms) from contacting or pressing on the eye while the wearer is sleeping. The eye mask can thus potentially avoid acute increases in the pressure in the wearer's eyes caused by wearing eyewear to sleep with or from objects pressing on the eye during sleep.

The eye mask can be made to be comfortable so that a wearer's sleep is not disturbed. The eye mask can be made to not cause an inordinate amount of disturbance to a wearer's hair or eye make-up. The eye mask can be shaped in a way that minimizes indentation marks on the facial skin. The eye mask can be made with materials that block out light.

In one embodiment, the eye mask is made of materials all of which are lightweight. In another embodiment, the eye mask is made to permit ventilation to avoid heat and moisture retention and to keep the wearer from becoming overly warm or uncomfortable. In another embodiment, the eye mask is comfortable enough that a wearer can use it during sleep for a long term of a week or more.

The fabric structure can be in the shape of roughly a FIG. 8 or a roughly a capital letter B or another shape that fits a wearer's face. An example of a shape is shown in FIG. 1 and FIG. 2. The fabric structure can have a first concave shape that fits over the left periorbital region of the wearer's face 20a and a second concave shape 20b that fits over the right periorbital region of the wearer's face. The first and second concave shape can be used to avoid having any portion of the eye mask, including the fabric of the fabric structure, contact the eyelid or eyelashes. In a preferred embodiment for the first and second concave support structures the concavity depth is 24.45 millimeters. In one embodiment, the fabric structure is shaped to wrap onto a portion of the temple. In one embodiment, the fabric structure is shaped to prevent movement of the mask while the wearer is sleeping. In one embodiment it has been found that the first and second support structures and the eye mask fabric, when extended over at least a portion of the temple, can help the eye mask stay in place while sleeping and make the eye mask more comfortable.

In one embodiment, the fabric structure can use a non-irritating and comfortable fabric on the portion of the fabric structure that contacts the wearer's face. This fabric can also be used for the entire fabric structure. The fabrics that can be used include but are not limited to well-known fabrics used for clothing or materials that regularly contact skin, including but not limited to wool, silk, polyester, nylon, cotton and blends of such fabrics. In one embodiment, the fabric can be of a color or contain a design that is attractive or whimsical. In some embodiments, the fabric structure contains one or more layers. In a preferred embodiment, the fabric structure contains two layers, one on each side of the first and second concave support structures.

In one embodiment, the fabric structure can also include padding. This padding can be made of a foam cushion or other material of natural origin, man-made origin or a combination of both. In one embodiment, this padding lies between each surface of the first and second concave support structures and the fabric structure. In another embodiment the padding is wrapped around the first and second concave support structures. In another embodiment, this padding is attached to the edges of the first and second concave support structures and it covers the first and second concave support structures. In another embodiment the padding is heat pressed to the inside of the fabric structure. In another embodiment the padding is glued to the inside of the fabric structure. In another embodiment the fabric structure and padding are bonded together thereby eliminating a distinct layer of padding.

In one embodiment, the fabric structure can contain holes for ventilation and moisture evaporation to aid in keeping the wearer comfortable. In one embodiment, the fabric is woven in a manner that provides holes for ventilation and moisture evaporation. In another embodiment, the fabric for the fabric structure is chosen so as to avoid heat retention under the mask and to maintain comfort. In another embodiment the fabric is light blocking. In another embodiment, any edges of the fabric structure that contact a wearer's face are made to be soft or otherwise not be sharp or pointed to minimize discomfort of the wearer. In one embodiment, the fabric structure contains from limited amounts to no stitching that could potentially irritate a wearer.

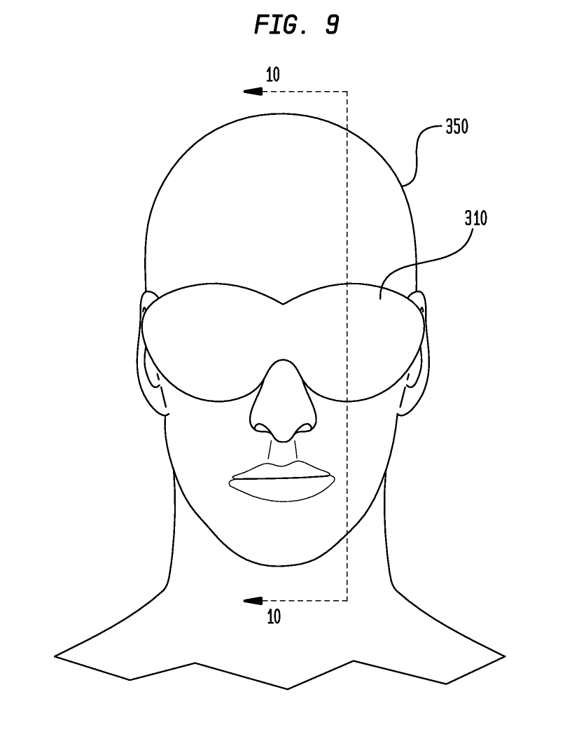

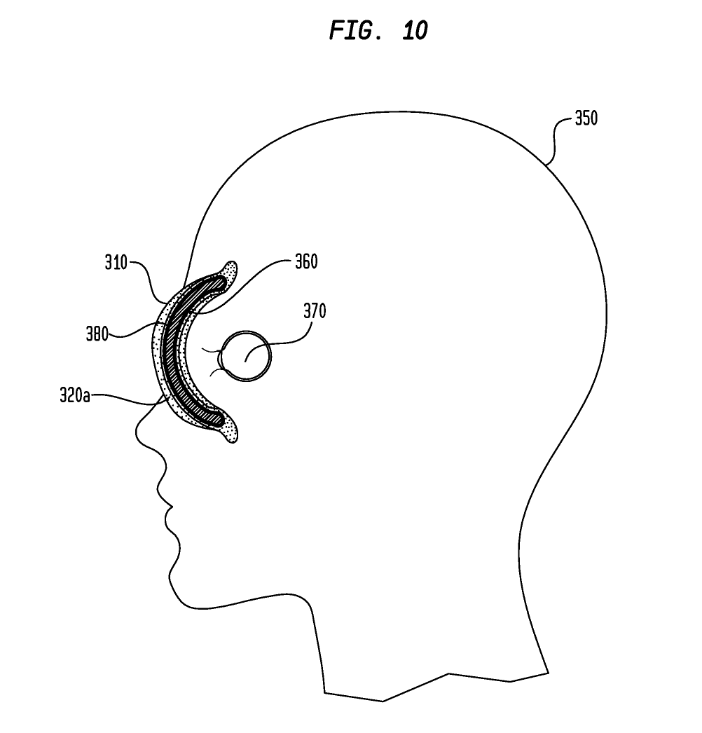

In one embodiment the fabric structure ends at the perimeter of the first and second concave support structures while totally encasing them. In another embodiment the fabric structure extends further than the perimeter of the first and second concave support structures, which can potentially provide for additional light blocking and comfort. An example of this latter feature of an embodiment is shown in FIG. 9 and FIG. 10.

The eye mask can also include a strap structure that is attached to the fabric structure and which holds the eye mask on the wearer's head. In one embodiment, the strap structure is adjustable. In another embodiment, the strap structure is self-adjusting by using, for example, an elastic portion for all or part of the strap structure. In one embodiment the strap structure length is adjustable by the use of sliding structures that can move along the strap structure and adjust its length. An example of a sliding structure is shown in FIG. 1 as two sliders 25 (each slider can be made of plastic, metal or other appropriate material) that receive portions of the strap structure 30 and which when moved closer together lengthens the strap and when moved farther apart tightens the strap. In one embodiment, the strap structure can be wider or narrower depending on the wearer's particular head shape and size. In one embodiment, the strap structure can comprise more than one portion that fits around a wearer's head.

The strap structure can be attached to the fabric structure by various means at one or more points. In one embodiment, the strap structure is attached by sewing. In another embodiment, the strap structure is attached by gluing. In another embodiment, the strap structure is attached with a grommet or other device that allows the strap structure to rotate or otherwise move. In another embodiment, the strap structure is attached by heat pressing. In another embodiment, the strap structure is fed through loops in the fabric structure at the temple edges of the fabric structure. In one embodiment, the strap structure is removable and replaceable. In another embodiment, the strap structure is attached with a hook and loop fabric fastener (e.g., Velcro). In another embodiment, the strap structure is attached with snaps.

The first and second concave support structures can be made of any rigid or semi-rigid material. This includes rubber, metal, nylon, polycarbonate, plastics, but also other materials. In one embodiment, the material is chosen to be lightweight so as to add to the comfort of the eye mask. In one embodiment, the first and second concave support structures contain one or more holes or gaps for ventilation and moisture evaporation to aid in keeping the wearer comfortable. The holes should be large enough to permit at least some ventilation and moisture evaporation but not so large as to significantly reduce the support provided to the fabric structure (e.g., the holes should not be so large as to permit the fabric structure to rest on the eyelid) or allow a finger to contact the eyelid. In another embodiment, any edges of the first and second concave support structures are made to be soft or malleable or otherwise not be sharp, pointed, or blunt edged, which if adopted can potentially minimize the discomfort of the wearer. In one embodiment, the first and second concave support structures contain one or more holes or have a non-solid structure to reduce their weight.

The first and second concave support structures should be at least firm enough so that the eye mask does not contact the eyelids or eyelashes when the eye mask is worn. The first and second concave support structures should also be firm enough so that when objects (e.g., mattresses, sheets, blankets, pillows, fingers, hands, arms) contact the eye mask, as is common while a person is sleeping, the first and second support structures do not significantly change shape and thus the eye mask will not contact the eyelids or eyelashes. When the eye mask is worn while sleeping, forces from objects should be transferred to the periorbital regions of the face and not the eyeballs, eyelids or eyelashes. In a preferred embodiment, the first and second concave support structures are firm enough that considerable force or pressure (e.g., force or pressure resulting from a greater than 5 lbs. weight pressing on an eye) from objects is resisted and the eye mask does not contact the eyelids or eyelashes when such force is applied. Several possible methods of evaluation and variations thereof can be used to examine an eye mask to determine whether it proffers an appropriate fit and sufficient protection against objects and the eye mask contacting the eye while the wearer is sleeping. These include:

a. the eye mask when worn should rest on the periorbital regions of the face and not contact the eyelashes or eyelids of the wearer.

b. the eye mask should be of sufficient strength that force or pressure from mattresses, sheets, blankets, pillows, fingers, hands and arms, which are commonly encountered while sleeping, will not cause the eye mask to contact the eyelashes or eyelid of the wearer.

c. a metal weight with a flat surface (e.g., a 5 lb. barbell weight) that is placed against both concave support shapes of an eye mask, while the eye mask is laying flat against a table (inside surface of the eye mask down), will not deform the eye mask to such an extent that the concave support shapes of the eye mask bend and touch the table.

If an eye mask performs acceptably in evaluations such as these, then the eye mask should provide appropriate fit and sufficient protection against objects and the eye mask contacting the eye while the wearer is sleeping.

The concave support structures can be shaped to fit the face around each eye separately. In this embodiment, there will be a first concave support structure for the left eye and a second concave support structure for the right eye. In this embodiment, the fabric structure has a first concave shape that fits over the left periorbital region of the wearer's face and a second concave shape that fits over the right periorbital region of the wearer's face. In this embodiment, the first concave support structure is placed inside the fabric structure in the first concave shape and the second concave support structure is placed inside the fabric structure in the second concave shape.

An example of an embodiment of an eye mask 310 and how it can fit a wearer's head 350 is shown in FIG. 9. As shown in FIG. 10, the eye mask 310 of this embodiment while on the wearer's head 350, including the inside surface 360 and the outside surface 380 of the first concave shape 320a, do not contact the eye 370.

In one embodiment, the first and second concave support structures are shaped to fit an individual wearer's face. Thus, custom concave support structures can be made for each wearer or a group of similar people with similar facial sizes and features. Measurements of the individual wearer's periorbital region can be taken to shape the concave support structures. Alternatively, a mold of the individual wearer's periorbital region using wax or clay or some other malleable material can be made and used to shape the concave support structures, by, for example, using the mold to create the reverse of the mold and form a shape for a concave support structure. In another embodiment, photographs or measurements of a wearer's face are entered into a Computer Assisted Design program and used to fit the concave support structures. In another embodiment, custom concave support structures can be made by laser scanning of a wearer's periorbital regions and making the concave support structures to conform to the scanned images. In another embodiment laser scanned images of a wearer's periorbital regions are uploaded to a 3D printer to produce custom support structures.

In one embodiment, an average of different wearer's periorbital region measurements are used to create a shape for the concave support structures that fit at least many if not all eyes. In another embodiment, photographs or measurements from several periorbital regions are entered into a Computer Assisted Design program and a population characteristic is determined (e.g., an average) and used to shape the concave support structures to all or subsets of the population that was evaluated. In another embodiment, various sizes of concave support structures are made available to provide a close fit for individual wearers. In another embodiment, once the shape of the concave support structures is determined, they are made with 3D printing. In another embodiment multiple concave support structures are made using injection moldings.

The first and second concave shapes of the eye mask can be made to have a particular shape, and, for example, fit an individual wearer's face, or a group of wearer's faces, as described herein for the first and second concave support structures.

In one embodiment, all of the materials chosen for the eye mask are lightweight. In one embodiment, the materials are chosen so that they allow some air to circulate and some heat and moisture to escape. In another embodiment, the eye mask is washable.

Eye mask embodiments can be worn by pulling on the strap structure and fitting the fabric structure over the eyes and the strap structure over the back of the head. In some embodiments, the eye mask is comfortable enough to be worn all night while the wearer is sleeping. In some embodiments, the eye mask is comfortable enough to be worn every night for a long term of a week or more. In some embodiments the eye mask fabric structure and the first and second concave support structures contain holes allowing for limited visibility without removal of the eye mask. In another embodiment, the eye mask fabric structure and the first and second concave support structures are transparent to let some light in and/or to permit at least some visibility.

The components of eye mask embodiments can be manufactured by several different methods depending on the design and materials used. These methods include but are not limited to the cutting of the fabric structure out of a larger piece of one or more fabrics and pieces joined together if necessary with sewing, gluing, heat, pressure, or other means or combinations of means (e.g., heat and pressure). In embodiments containing padding, the padding can be cut from a larger piece of one or more materials and joined together if necessary with sewing, gluing, heat, pressure or other means or combinations of means (e.g., heat and pressure). In another embodiment, the padding is part of the fabric structure. In embodiments with concave support structures, the concave support structure can be cut out of a larger piece of one or more materials and shaped, if necessary, using heat, pressure, injection molding, or other means or combinations of means (e.g., heat and pressure), into the desired dimensions and cut or joined together with other pieces if necessary to form a finished concave support structure. In another embodiment of an eye mask, fabric, cushioning or padding material, and a support material are layered and then molded together to shape the eye mask.

In some embodiments, the concave support structure is made by a 3D printer. An “L” and “R” label 282a, 282b can be optionally added to the left and right concave support structures as shown, for example, in FIG. 5, FIG. 6, and FIG. 7, which can assist in their correct placement in the eye mask. In one embodiment, the concave support structures are made by melting plastic and forming it into concave support structures using a mold. In a preferred embodiment concave support structures are made using injection moldings. The strap structure can be attached to the fabric structure by several means, including straps, grommets, rivets, glue, snaps, buttons, heat, pressing, threading through loops in the fabric or attached to the fabric, and combinations of these. All of these components can be made by different means than what is described here.

Once made, the components of the eye mask (e.g., fabric structure, optional padding, concave support structures, strap structure, and other components, depending on which of these components are used) can be fitted or joined together with sewing, gluing, heat, pressure, or other means or combinations of means (e.g., heat and pressure). In some embodiments, the padding is wrapped around or attached to the edges of the concave support structures before the components are fitted or joined together. In other embodiments, the padding is attached on the insides of the fabric structure before the components are fitted or joined together. In other embodiments, components of the eye mask are made and formed at the same time and joined together with heat and pressure. There are additional ways to form an eye mask than what is described here.

In one specific example, the eye mask is composed of five layers in a cross-section taken of the portion of the mask that is over the eyes. From inside (near the face) to outside, the layers are: an inside layer of the fabric structure; a first padding layer; a concave support structure layer; a second padding layer; and an outside layer of the fabric structure. The eye mask is manufactured by heat pressing, molding, gluing, or stitching these components together and there is no stitching on the facial side of the eye mask in order to avoid irritating the skin. The fabric structure uses a fabric that is comfortable and non-irritating. The fabric structure has a first concave shape that fits over the left periorbital region of the wearer's face and a second concave shape that fits over the right periorbital region of the wearer's face. The fabric structure may contain holes or be of a loose weave for ventilation and moisture evaporation to aid in keeping the wearer comfortable. The fabric structure and the other materials used in the eye mask are chosen to avoid heat retention under the mask to maintain comfort. The fabric structure and first and second concave support structures contained within wrap onto the temple as well as the cheeks, brow and nose to avoid slipping and discomfort while sleeping. The eye mask blocks out light, is lightweight so as to not unnecessarily weigh on the nose bridge or create lines on the skin for most wearers. The edges of the eye mask that contact the skin are not pointed or sharp so as to avoid pressing into the skin. The edges of the concave support structures are also not pointed or sharp to aid in comfort. The concave support structures are shaped by averaging the measurements taken from laser scanning of the periorbital structures of several different sizes of faces to make them an anatomically good fit. The concave support structures are made of nylon and made by 3D printing. The strap structure is attached at two points to the fabric structure and it is a narrow, elastic band that fits around the head and contributes to comfort while securing the mask on the head without causing an inordinate amount of disorder to some wearers' hair or make-up. The band is adjustable using a sliding structure. The eye mask is washable, which contributes to its usefulness for long-term use for more than a week or more.

In another specific example, an eye mask for sleeping is provided that in cross section has a first layer of fabric, an optional layer of padding, a layer of plastic (that provides support as described regarding the first and second concave structures described herein) and a second layer of fabric. These layers are shaped and joined together to form a portion of an eye mask that has a first concave shape that fits over the left periorbital region of a wearer and a second concave shape that fits over the right periorbital region of the wearer. This example also has a strap structure that holds the eye mask on the wearer's head. When this embodiment is worn, the eye mask does not contact the left or right eyelids or eyelashes, the eye mask is worn comfortably on the wearer's head while sleeping, and the eye mask protects the wearer's eyes from objects pressing against the eyes. Ventilation can be provided in the first layer of fabric, the optional layer of padding, the layer of plastic and/or the second layer of fabric by using materials that permit the passage of heat and moisture. The comfort of the eye mask of this embodiment can be enhanced by having the eye mask extend over an appropriate portion of the temple.

This latter example can be manufactured by molding and joining (e.g., simultaneously molding and joining the layers by using heat and pressure) the first layer of fabric, the optional layer of padding, the layer of plastic and the second layer of fabric to form a portion of an eye mask that has a first concave shape that fits over the left periorbital region of a wearer and a second concave shape that fits over the right periorbital region of the wearer. A strap structure is then attached to the portion of the eye mask. With this embodiment and the other embodiments described herein, it is possible to add additional components and layers to the eye mask to achieve results consistent with those described herein, including but not limited to, protection against objects contacting the eye, decoration, ventilation and comfort.

Those skilled in the art will recognize that many embodiments are possible within the scope of the invention. Variations, modifications and combinations of the various parts described herein and otherwise applied by a person skilled in the art can be made and still fall within the scope of the invention.

I claim:

1. An eye mask for sleeping comprising:
a fabric structure that fits over both eyes of a wearer and which has a first concave shape that fits over the left periorbital region of the wearer and a second concave shape that fits over the right periorbital region of the wearer,
a first concave support structure that fits inside the first concave shape of the fabric structure,
a second concave support structure that fits inside the second concave shape of the fabric structure,
a strap structure that is attached to the fabric structure and which is of a length to wrap around the wearer's head so as to hold the eye mask on the wearer's head, and
wherein when worn, the first concave support structure and the second concave support structure are firm enough and fit inside the fabric structure so that the eye mask does not contact the left or right eyelids or eyelashes, the eye mask is worn comfortably on the wearer's head while sleeping, and the eye mask protects the wearer's eyes from objects pressing against the eyes.

2. The eye mask of claim 1 wherein there is padding between the first concave support structure and the fabric structure and the second concave support structure and the fabric structure.

3. The eye mask of claim 1 wherein the fabric structure comprises a fabric that permits at least some ventilation.

4. The eye mask of claim 1 wherein the first and second concave support structures permit ventilation.

5. The eye mask of claim 1 wherein the fabric structure and the first and second concave support structures wrap onto a portion of the wearer's temples.

6. The eye mask of claim 1 wherein the strap structure is a narrow elastic band.

7. The eye mask of claim 1 wherein the strap structure is adjustable so it can be tightened or loosened.

8. The eye mask of claim 1 that is comfortable so that it can be worn during sleep every night for more than a week without irritating the wearer's nose, brow, cheek or skin.

9. The eye mask of claim 1 that is light blocking.

10. The eye mask of claim 1 that is at least partially transparent.

11. An eye mask for sleeping comprising:
a fabric structure comprising a fabric that permits at least some ventilation, said fabric structure fits over both eyes of a wearer and wraps onto portions of the wearer's temple and said fabric structure has a first concave shape that fits over the left periorbital region of the wearer and a second concave shape that fits over the right periorbital region of the wearer,
a first concave support structure that fits inside the first concave shape of the fabric structure,
a second concave support structure that fits inside the second concave shape of the fabric structure,
padding between the first concave support structure and the fabric structure and the second concave support structure and the fabric structure,
wherein the first concave support structure and padding is contained in the fabric structure so that when worn, the eye mask does not contact the left eyelid or eyelashes,
wherein the second concave support structure and padding is contained in the fabric structure so that when worn, the eye mask does not contact the right eyelid or eyelashes,
a strap structure that is elastic, attached at two points to the fabric structure, and which is of a length to wrap around the wearer's head so as to hold the eye mask on the wearer's head, and
the eye mask is worn comfortably on the wearer's head while sleeping and it protects the wearer's eyes from objects pressing against the eyes.

12. A method of manufacturing an eye mask for sleeping comprising:
constructing a multilayer fabric structure with a first concave shape that fits over the left periorbital region of a wearer and a second concave shape that fits over the right periorbital region of a wearer,
molding a first concave support structure using injection molding,
molding a second concave support structure using injection molding,
inserting the first concave support structure into the first concave shape of the fabric structure,
inserting the second concave support structure into the second concave shape of the fabric structure,
joining the inside and outside of the fabric structure together after inserting the first and second concave support structures, and
attaching a strap structure to the fabric structure.

13. The method of claim 12 further comprising;
inserting padding between the first concave support structure and the fabric structure, and
inserting padding between the second concave support structure and the fabric structure.

14. A method of protecting the eyes from objects putting pressure on the eyes while sleeping, said method comprising:
wearing an eye mask while sleeping, said eye mask comprising;
a fabric structure comprising a fabric that permits at least some ventilation, said fabric structure fits over both eyes of a wearer and wraps onto portions of the wearer's temple and said fabric structure has a first concave shape that fits over the left periorbital region of the wearer and a second concave shape that fits over the right periorbital region of the wearer,
a first concave support structure that fits inside the first concave shape of the fabric structure,
a second concave support structure that fits inside the second concave shape of the fabric structure,
padding between the first concave support structure and the fabric structure and the second concave support structure and the fabric structure,
wherein the first concave support structure and padding is contained in the fabric structure so that when worn, the eye mask does not contact the left eyelid or eyelashes,
wherein the second concave support structure and padding is contained in the fabric structure so that when worn, the eye mask does not contact the right eyelid or eyelashes,
a strap structure that is elastic, attached at two points to the fabric structure, and which is of a length to wrap around the wearer's head so as to hold the eye mask on the wearer's head, and
the eye mask is worn comfortably on the wearer's head while sleeping and it protects the wearer's eyes from objects pressing against the eyes.

15. An eye mask for sleeping comprising,
a first layer of fabric, a layer of plastic and a second layer of fabric that are shaped and joined to form a portion of an eye mask that has a first concave shape that fits over the left periorbital region of a wearer and a second concave shape that fits over the right periorbital region of the wearer;
a strap structure; and
when worn the eye mask does not contact the left or right eyelids or eyelashes, the eye mask is worn comfortably on the wearer's head while sleeping, and the eye mask protects the wearer's eyes from objects pressing against the eyes.

16. The eye mask for sleeping of claim 15 further comprising a layer of padding between the first layer of fabric and the layer of plastic.

17. A method of manufacturing an eye mask for sleeping comprising:
molding and joining a first layer of fabric, a layer of plastic and a second layer of fabric to form a portion of an eye mask that has a first concave shape that fits over the left periorbital region of a wearer and a second concave shape that fits over the right periorbital region of the wearer;
attaching a strap structure to the portion of an eye mask; and
when worn the eye mask does not contact the left or right eyelids or eyelashes, the eye mask is worn comfortably on the wearer's head while sleeping, and the eye mask protects the wearer's eyes from objects pressing against the eyes.

18. The method of manufacturing of claim 17 wherein the molding and joining step further comprises a layer of padding between the first layer of fabric and the layer of plastic.
